(19) United States
(12) Patent Application Publication
(10) Pub. No.: US 2020/0288951 A1
(43) Pub. Date: Sep. 17, 2020

(54) ENDSCOPE PORT STOPPER

(71) Applicant: 3M INNOVATIVE PROPERTIES COMPANY, SAINT PAUL, MN (US)

(72) Inventors: GUENTER M. ZILLIGEN, GREVENBROICH (DE); HENNING REUTER, WILLICH (DE); G. MARCO BOMMARITO, STILLWATER, MN (US)

(73) Assignee: 3M INNOVATIVE PROPERTIES COMPANY, SAINT PAUL, MN (US)

(21) Appl. No.: 16/653,000

(22) PCT Filed: Sep. 18, 2018
(86) PCT No.: PCT/US2018/051476

§ 371 Date: Apr. 2, 2020

Related U.S. Application Data
(60) Provisional application No. 62/567,522, filed on Oct. 3, 2017.

(51) Int. Cl. A61B 1/00; A61B 1/12

(52) U.S. Cl. CPC A61B 1/00137; A61B 1/00068; A61B 1/12

(57) ABSTRACT

Provided is an endoscope port stopper including a first plug comprising a first central axis and a second plug comprising a second central axis, where the first plug is configured to releasably seal a first endoscope port and the second plug is configured to releasably seal a second endoscope port. In some embodiments, port stoppers may further include a third plug and/or a fourth plug.

### TECHNICAL FIELD

This disclosure relates in general to the field of medical instrument port stoppers and more particularly to an apparatus for stoppering an endoscope port to create a seal between the interior of the endoscope and the outside environment.

### BACKGROUND

Millions of gastrointestinal endoscopy procedures are performed on an annual basis around the world. These endoscopy procedures are typically performed using complex, flexible instruments that, when inserted into a patient's body, may become contaminated with biomaterial and microorganisms, including potential pathogens. Thus, careful and thorough cleaning of flexible endoscopes between patients is critical to reducing the risk of cross-contamination and the possible transmission of pathogens during an endoscopy procedure.

In the process of cleaning or evaluating the cleanliness of an endoscope, it may be desirable to flush the interior of the endoscope with one or more fluids, e.g., water, air. But, to control how a fluid enters and exits an endoscope, selective stoppering of one or more ports of the endoscope may be required.

### SUMMARY

In one aspect, provided is an endoscope port stopper comprising a first plug including a first central axis, and a second plug comprising a second central axis, where the first plug is configured to releasably seal a first endoscope port and the second plug is configured to releasably seal a second endoscope port.

In another aspect, provided is an endoscope port stopper comprising a first plug including a first central axis, a second plug comprising a second central axis, and a third plug comprising a third central axis, where the first plug is configured to releasably seal a first endoscope port, the second plug is configured to releasably seal a second endoscope port, and the third plug is configured to releasably seal a third endoscope port.

In another aspect, provided is an endoscope port stopper comprising a first plug including a first central axis, a second plug comprising a second central axis, a third plug comprising a third central axis, and a fourth plug comprising a fourth central axis, where the first plug is configured to releasably seal a first endoscope port, the second plug is configured to releasably seal a second endoscope port, the third plug is configured to releasably seal a third endoscope port, and the fourth plug is configured to releasably seal a fourth endoscope port.

Features and advantages of the present disclosure will be further understood upon consideration of the detailed description as well as the appended claims.

### BRIEF DESCRIPTION OF THE DRAWINGS

FIG. 1 is a side perspective view of a first embodiment of a stopper of the present disclosure.

FIG. 2 is a side elevational view of the embodiment of FIG. 1.

FIG. 3 is a side sectional view of the embodiment of FIG. 1.

FIG. 4 is a detail of a portion of the side sectional view of the embodiment of FIG. 1.

FIG. 5 shows an endoscope control body having ports of different dimensions that may be releasably sealed by the stopper of FIG. 1.

FIG. 6 is a plan view of a second embodiment of a stopper of the present disclosure.

FIG. 7 is a plan view of a third embodiment of a stopper of the present disclosure.

FIG. 8 is a plan view of an embodiment of a plug of the present disclosure.

FIG. 9 is a plan view of an embodiment of a plug of the present disclosure.

FIG. 10 is a plan view of an embodiment of a plug of the present disclosure.

FIG. 11 is a plan view of an embodiment of a plug of the present disclosure.

FIG. 12 is a side elevational view of a fourth embodiment of a stopper of the present disclosure.

FIG. 13 is a side sectional view of the first embodiment of a stopper of FIG. 1 with a cavity in an end of a plug.

Repeated use of reference characters in the specification and drawings is intended to represent the same or analogous features or elements of the disclosure. It should be understood that numerous other modifications and embodiments can be devised by those skilled in the art, which fall within the scope and spirit of the principles of the disclosure. The figures may not be drawn to scale.

### DETAILED DESCRIPTION

Flushing an endoscope with sample fluid, e.g. sterile water, saline, special recovery fluids (such as broth), and collection of the fluid sample as it exits the endoscope can provide the endoscope operator with the opportunity to test the fluid sample for residual contaminants e.g., ATP, protein, microbial contamination, that may remain in the endoscope interior after reprocessing, such as cleaning and/or disinfection, thus providing a measure for cleanliness and/or disinfection efficacy of endoscope reprocessing methods and operations.

Because an endoscope may include ports along its length that allow for fluids, e.g., water, air, to both enter and exit the interior of the device, it may be desirable to close at least some of these ports e.g., a suction valve opening (“SVO”), an instrument or biopsy channel port, an air/water valve opening, prior to flushing the endoscope with a chosen fluid to prevent the fluid from leaking from the device at the location of such ports. To prevent the undesired fluid leakage, endoscope ports may be reversibly sealed, for example, with an appropriate stopper. However, as shown by the data in Table 1, because endoscope port dimensions, e.g., diameter of the opening on the outside of the endoscope, depth of the port, diameter of the opening on the interior of the endoscope where the port intersects a channel, may vary according to their location on the device and by manufacturer, more than one stopper configuration may be required to seal different ports on the same endoscope.

TABLE 1
Port Dimensions for Endoscopes from Different Manufacturers

| Port | OLYMPUS | | FUJI (SVO is stepped) | | | | PENTAX | |
|---|---|---|---|---|---|---|---|---|
| Dimensions (mm) | Diameter | Depth | Diameter 1 | Depth 1 | Diameter 2 | Depth 2 | Diameter | Depth |
| Suction valve | 5.6 | 22.6 | 9.5 | 4.9 | 6.2 | 20.8 | 8.3 | 27 |
| opening | | | | | | | | |
| (“SVO”) | | | | | | | | |
| Instrument | 5.9 | 46.2 | 5.9 | 53.8 | — | — | 4.1 | 54.6 |
| or biopsy | | | | | | | | |
| channel | | | | | | | | |

Furthermore, if a stopper extends too far into the interior of the endoscope when sealing the port, for example, greater than 20.8 mm on the FUJI SVO, greater than 22.6 mm on the OLYMPUS SVO, or greater than 27 mm on the PENTAX SVO, the flow of fluid through the endoscope channel may be restricted or entirely prevented. Therefore, it would be beneficial to provide the endoscope operator with a single endoscope port stopper that is both easy to insert and remove, and capable of sealing a variety of openings, while not restricting the flow of fluid through the endoscope interior when the opening is stoppered.

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the term “coupled” and variations thereof are used broadly and encompass both direct and indirect couplings. It is to be understood that other embodiments may be utilized, and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

Provided in the present disclosure is an endoscope port stopper including a first plug comprising a first central axis and a second plug comprising a second central axis, wherein the first plug is configured to releasably seal a first endoscope port and the second plug is configured to releasably seal a second endoscope port.

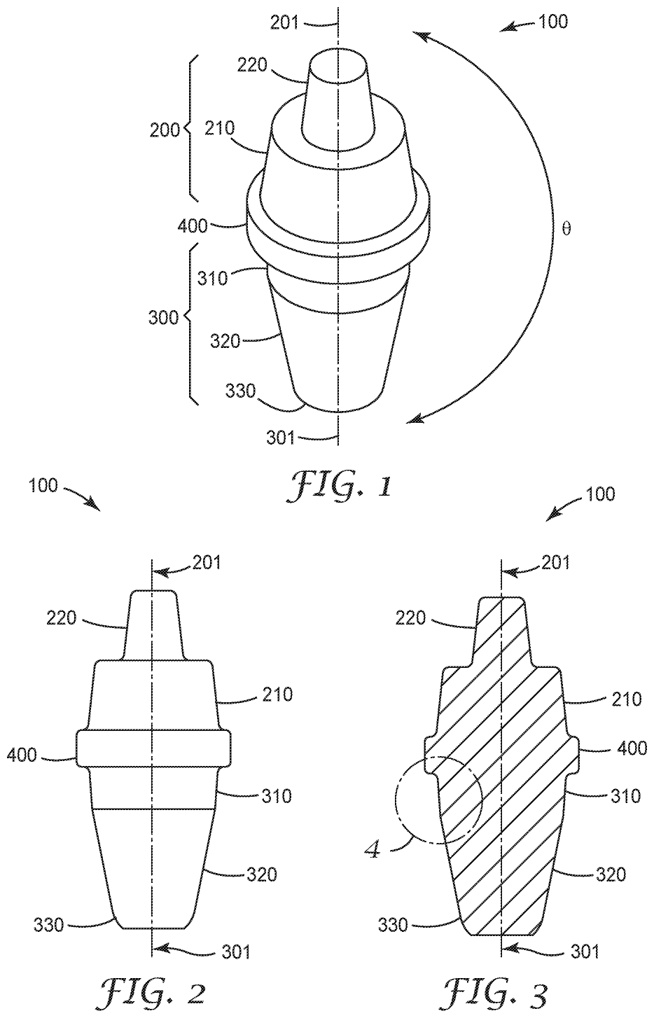

FIGS. 1-3 illustrate an endoscope port stopper 100 according to one embodiment of the present disclosure. As shown in FIGS. 1-3, the port stopper 100 has a first plug 200 comprising a first central axis 201 and a second plug 300 comprising a second central axis 301, where the first plug 200 is configured to releasably seal a first endoscope port, the first endoscope port having a first set of dimensions, such as, for example, a 4.1 mm diameter and a 54.6 mm depth, and the second plug 300 is configured to releasably seal a second endoscope port, either on the same endoscope or on a different endoscope, having a second set of dimensions, such as, for example, an 8.3 mm diameter and a 27 mm depth.

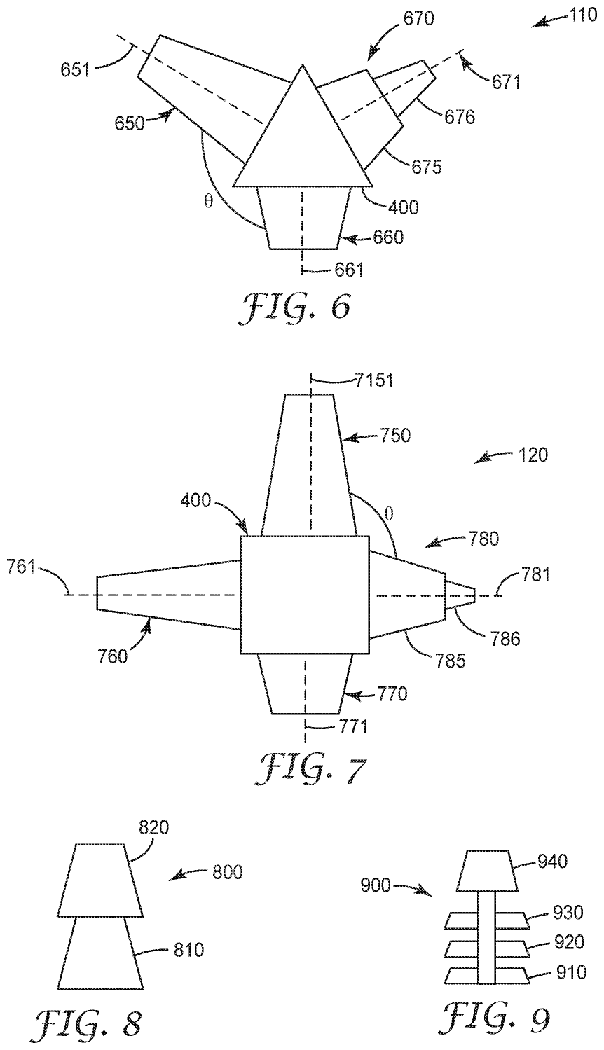

In FIGS. 1-3, the first central axis 201 and the second central axis 301 have an angle θ between them of about 180°. As shown in FIGS. 6 and 7, other values of angle θ are contemplated for different stopper 110, 120 embodiments. For a single stopper, angle θ can be equal between each plug or can be different between each plug. Referring to FIG. 6, in some embodiments, a stopper 110 may include a first plug 650 comprising a first central axis 651, a second plug 660 comprising a second central axis 661, and a third plug 670 comprising a third central axis 671, where the angle θ between the central axes is about 120°. Referring to FIG. 7, in some embodiments, a stopper 120 may include a first plug 750 comprising a first central axis 751, a second plug 760 comprising a second central axis 761, a third plug 770 comprising a third central axis 771, and a fourth plug 780 having a fourth central axis 781, where the angle θ between the central axes is about 90°. Generally, angle θ may have values from 45° to 225° in embodiments of the present disclosure.

As shown in FIGS. 1-3, the first plug 200 can have a first plug first sealing region 210 having a generally frustoconical shape, and a first plug second sealing region 220 also having a generally frustoconical shape. In some embodiments, the smallest cross-sectional diameter of the first plug first sealing region 210 is greater than the largest cross-sectional diameter of the first plug second sealing region 220. In one embodiment, the first plug first sealing region 210 has a smallest cross-sectional diameter of 9.3 mm, a largest cross-sectional diameter of 10.4 mm, and a height of 5.7 mm and the first plug second sealing region 220 has a smallest cross-sectional diameter of 3.65 mm, a largest cross-sectional diameter of 4.84 mm, and a height of 5.7 mm.

The second plug 300 can have a second plug first sealing region 310 having a generally frustoconical shape, and a second plug second sealing region 320 also having a generally frustoconical shape, where the smallest cross-sectional diameter of the second plug first sealing region 310 is greater than or equal to the largest cross-sectional diameter of the first plug second sealing region 320. In one embodiment, the second plug first sealing region 310 has a smallest cross-sectional diameter of 9.6 mm, a largest cross-sectional diameter of 10.1 mm, and a height of 3 mm and the second plug second sealing region 320 has a smallest cross-sectional diameter of 6.4 mm, a largest cross-sectional diameter of 9.6 mm, and a height of 8.6 mm.

The second plug 300 can, as shown, further have a third sealing region, 330, also with a generally frustoconical shape, where the smallest cross-sectional diameter of the second plug second sealing region 320 is greater than or equal to the largest cross-sectional diameter of the second plug third sealing region 330. In one embodiment, the second plug second sealing region 320 has a smallest cross-sectional diameter of 6.4 mm, a largest cross-sectional diameter of 9.6 mm, and a height of 8.6 mm and the second plug third sealing region 330 has a smallest cross-sectional diameter of 4.4 mm, a largest cross-sectional diameter of 6.4 mm, and a height of 1.6 mm.

In some embodiments, and as shown in FIGS. 1-3, the smallest cross-sectional diameter of the second plug 300 may be greater than the smallest cross-sectional diameter of the first plug 200. In some embodiments, and as shown in FIGS. 1-3, the largest cross-sectional diameter of the first plug 200 may be greater than the largest cross-sectional diameter of the second plug 300.

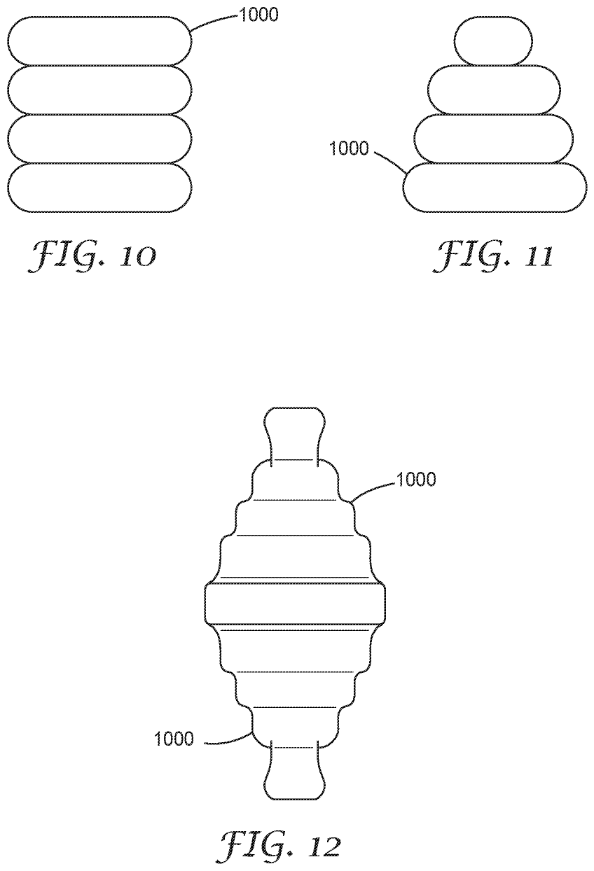

In some embodiments, a plug may include only one sealing region. As shown in FIG. 6, an embodiment of a stopper 110 includes plug 650 and plug 660 having only one sealing region, and in FIG. 7, a stopper 120 includes plug 750, plug 760, and plug 770 having only one sealing region. As shown in FIGS. 6-8, in some embodiments, a plug may have more than one sealing region. Referring to FIG. 6, a second embodiment of a stopper of the present disclosure, stopper 110 includes a plug 670 having two sealing regions 675, 676. Referring to FIG. 7, a third embodiment of a stopper of the present disclosure, stopper 120 includes a plug 780 having two sealing regions 785, 786. In some embodiments, all plugs on a stopper may include only one sealing region. In some embodiments, all plugs on a stopper may include two or more sealing regions. As shown in FIG. 8, a plug 800 may include two sealing regions 810, 820 having the same or similar shape. As shown in FIG. 9 and in some embodiments, a plug 900 may include more than two sealing regions 910, 920, 930, 940 having similar and/or different shapes. In some embodiments, the sealing region 1000 shape can be other than generally frustoconical, as shown in FIGS. 10-12.

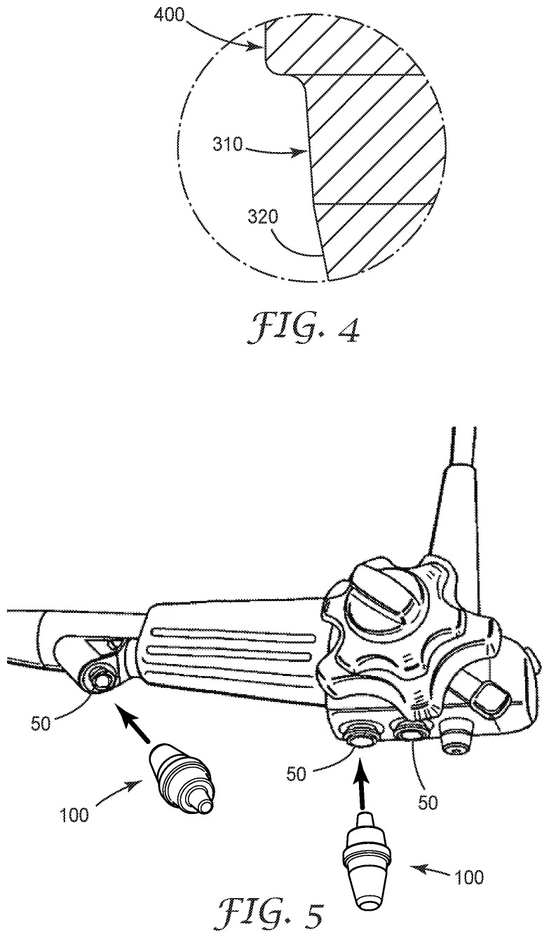

In some embodiments, and as shown in FIGS. 1-4, the stopper 100 may include a rim 400 positioned between, i.e., separating, the first plug 200 and the second plug 300, such that the rim 400 has a cross-sectional diameter greater than the greatest cross-sectional diameter of the first plug 200 and the second plug 300. In some conditions, the rim 400 can act to prevent the plug 200 or 300 from extending further into a port hole than desired by the operator and can also act to cover irregularities in the endoscope port opening, e.g., a groove in the circumference of the port that enlarges the port diameter at the location of the groove, to provide a water-tight seal of the port. In some embodiments, the rim 400 may have a cross-sectional diameter that is at least 0.8 mm, at least 1 mm, at least 2 mm, at least 3 mm, at least 4 mm, at least 5 mm, at least 6 mm, at least 7 mm, at least 8 mm, at least 9 mm, or at least 10 mm greater than the greatest cross-sectional diameter of the first plug 200 and the second plug 300. In some embodiments, the rim 400 may have a cross-sectional diameter that is no more than 20 mm, no more than 15 mm, no more than 10 mm greater than the greatest cross-sectional diameter of the first plug 200 and the second plug 300. In some embodiments, the cross-sectional diameter of the rim 400 may be 0.8 mm to 20 mm, 0.8 mm to 15 mm, 0.8 mm to 10 mm, 1 mm to 9 mm, 1 mm to 8 mm, 1 mm to 7 mm, 1 mm to 6 mm, 1 mm to 5 mm, 1 mm to 4 mm, 1 mm to 3 mm, or 1.5 mm to 2.5 mm, e.g., 1.9 mm, greater than the greatest cross sectional diameter of the first plug 200 and the second plug 300.

In some embodiments, the rim 400 may have a thickness, i.e., height of at least 1 mm, at least 1.5 mm at least 2 mm, or at least 2.5 mm. In some embodiments, the rim 400 may have a thickness of no more than 5 mm, no more than 4.5 mm, no more than 4 mm, or no more than 3.5 mm. In some embodiments, the rim 400 may have a thickness of 1 mm to 5 mm, 1.5 mm to 4.5 mm, 2 mm to 4 mm, or 2.5 mm to 3.5 mm, e.g., 3 mm.

Though shown as circular in FIGS. 1-3, other geometries for the rim 400, e.g., rectangular, square, trapezoidal, triangular, and irregular are possible. Moreover, and as shown in FIGS. 6 and 7, the rim 400 may be positioned between more than a first plug and a second plug. FIG. 6 shows an embodiment of stopper 110 having a first plug 650, a second plug 660, and a third plug 670 with the rim 400 separating each of the plugs 650, 660, 670. FIG. 7 shows an embodiment of stopper 120 having a first plug 750, a second plug 760, a third plug 770, and a fourth plug 780, with the rim 400 separating each of the plugs 750, 760, 770, 780.

A stopper of the present disclosure may be made of any suitably durable and conformable material. Suitable materials can include, for example, a silicone rubber, a neoprene rubber, a gum rubber, an ethylene propylene diene monomer (“EPDM”) rubber, a polyethylene rubber, a polypropylene rubber, a polyurethane foam rubber, a natural latex foam rubber, a polyethylene foam rubber, a polypropylene foam rubber, and combinations thereof. In some embodiments, the stopper 100 comprises a silicone rubber. Generally, the stopper may comprise a material that is stable when subjected to an ethylene oxide sterilization process.

A stopper of the present disclosure should be sufficiently conformable such that when it is inserted into an endoscope port, it fills at least a portion of the port, creating a water-tight seal, but also should be durable enough that it may be readily removed without breaking by an operator wishing to reopen the port. In some embodiments, the stopper may comprise a material having a Shore (A) hardness of 20-70, 25-60, 30-50, or 35-45, e.g., 40.

A rim, if included, may be formed of the same material as stopper or may be a different material. For example, the rim may be a harder or less conformable material as the plug portions of the stopper to further prevent the plug from extending further into a port hole than desired by the operator and can also act to cover irregularities in the endoscope port opening.

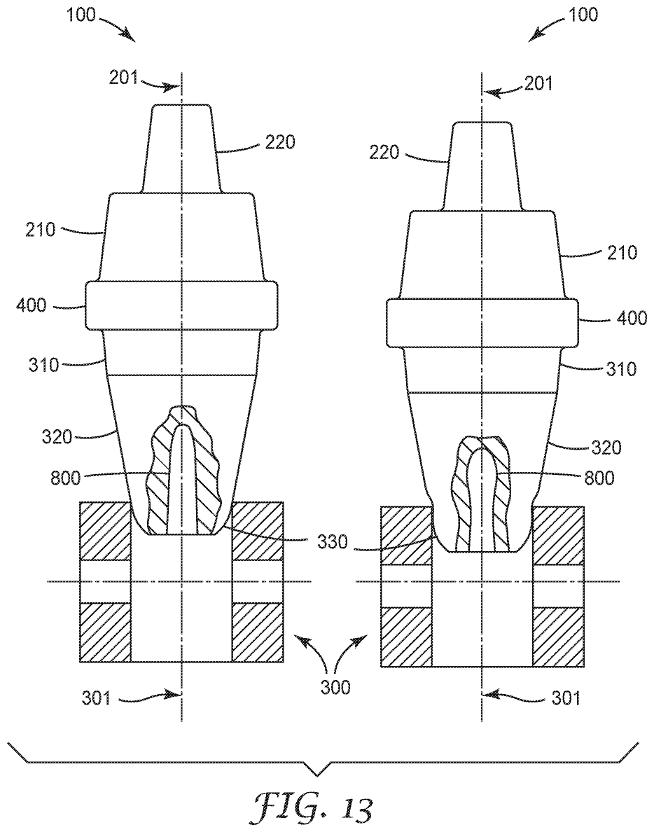

In some embodiments, and as shown in FIG. 13, any or all plugs of a stopper may comprise a cavity 800 in the plug. In FIG. 13, which is a side sectional view of an embodiment of FIG. 1, a cavity 800 is within the plug 300. The cavity 800 is a portion of the material of the plug removed to allow for flexibility of the sealing region of the plug. In FIG. 13, the cavity 800 is within the second plug sealing region 320. The cavity 800 is removed from the perimeter of the second plug sealing region 320 so that the perimeter of the second plug sealing region 320 remains intact. In one embodiment, a cavity 800 could extend entirely into any additional sealing regions of a plug. For example, in the embodiment shown in FIG. 13, the cavity 800 could extend in the first and second plug sealing region 310, 320. Any of the plugs in the embodiments disclosed could include a cavity in the sealing region.

Methods of making endoscope port stoppers of the present disclosure are well known to those of ordinary skill in the relevant arts, and may include, for example, conventional molding of a rubber or foam rubber to the desired specifications or three-dimensional printing techniques. In some embodiments, a stopper may be made of a unitary piece of material. In other embodiments, a stopper may be formed from two or more elements joined together, where the elements may be made from the same material or from different materials.

A stopper of the present disclosure may be used to releasably seal an endoscope port 50, as shown in FIG. 5, but could also be used to seal a port having appropriate dimensions on a different kind of device entirely.

To releasably seal an endoscope port with a stopper of the present disclosure, the operator may grasp the stopper by a first plug and insert a second plug into the endoscope port while pressing toward the body of the endoscope. In some embodiments, it may be desirable to press the second plug into the port while rotating the first plug, i.e., with a “screwing” motion. The stopper may be removed from the port when desired by grasping the first plug and pulling away from the body of the endoscope. Stoppers of the present disclosure may be useful for more than one port sealing, and it may therefore be desirable to clean and/or disinfect the stopper between uses, e.g., using an ethylene oxide sterilization process.

1. An endoscope port stopper comprising:
a first plug comprising a first central axis; and
a second plug comprising a second central axis,
wherein the first plug is configured to releasably seal a first endoscope port and the second plug is configured to releasably seal a second endoscope port.

2. The endoscope port stopper of claim 1, wherein an angle θ between the first central axis and the second central axis is 45° to 225°.

3. The endoscope port stopper of claim 2, wherein the angle θ is 180°.

4. The endoscope port stopper of claim 1, wherein the first plug comprises a first plug first sealing region and a first plug second sealing region, wherein the smallest cross-sectional diameter of the first plug first sealing region is greater than the largest cross-sectional diameter of the first plug second sealing region.

5. The endoscope port stopper of claim 1, wherein the second plug comprises a second plug first sealing region and a second plug second sealing region, wherein the smallest cross-sectional diameter of the second plug first sealing region is greater than the largest cross-sectional diameter of the second plug second sealing region.

6. The endoscope port stopper of claim 5, wherein the second plug further comprises a second plug third sealing region, wherein the smallest cross-sectional diameter of second plug second sealing region is greater than the largest cross-sectional diameter of the second plug third sealing region.

7. The endoscope port stopper of claim 4, wherein the largest cross-sectional diameter of the second plug second sealing region is less than or equal to the largest cross-sectional diameter of the first plug first sealing region.

8. The endoscope port stopper of claim 4, wherein at least one of the first plug first sealing region and the first plug second sealing region has a generally frustoconical shape.

9. The endoscope port stopper of claim 5, wherein at least one of the second plug first sealing region and the second port second sealing region 320 has a generally frustoconical shape.

10. The endoscope port stopper of claim 6, wherein the second plug third sealing region has a generally frustoconical shape.

11. The endoscope port stopper of claim 1, wherein the smallest cross-sectional diameter of the second plug is greater than the smallest cross-sectional diameter of the first plug.

12. The endoscope port stopper of claim 1, further comprising a rim, wherein the rim 400 is positioned between the first plug and the second plug.

13. The endoscope port stopper of claim 12, wherein the rim has a cross-sectional diameter greater than the greatest cross-sectional diameter of the first plug and the second plug.

14. The endoscope port stopper of claim 1, wherein the stopper comprises at least one of a silicone rubber, a neoprene rubber, a gum rubber, an ethylene propylene diene monomer (“EPDM”) rubber, a polyethylene rubber, a polypropylene rubber, a polyurethane foam rubber, a natural latex foam rubber, a polyethylene foam rubber, a polypropylene foam rubber, and combinations thereof.

15. (canceled)

16. (canceled)

17. The endoscope port stopper of claim 16, wherein at least one of the first plug and the second plug has a Shore (A) hardness of 40.

18. The endoscope port stopper of claim 1, further comprising a third plug comprising a third central axis, wherein the third plug is configured to releasably seal a third endoscope port.

19. (canceled)

20. The endoscopic port stopper of claim 1, further comprising a cavity within the first plug.

21. The endoscopic port stopper of claim 4, further comprising a cavity within the first plug first sealing region.

22. The endoscopic port stopper of any of claim 21, where in the cavity is with the first plug first sealing region and the first plug second sealing region.

23. The endoscopic port stopper of claim 1, further comprising a cavity within the first plug and second plug.

24. (canceled)

25. (canceled)
